(19) United States
(12) Patent Application Publication
(10) Pub. No.: US 2017/0151447 A1
(43) Pub. Date: Jun. 1, 2017

(54) Graphene Based Ultrasound Generation

(71) Applicant: BRAGI GmbH, Munchen (DE)

(72) Inventor: Peter Vincent Boesen, Munchen (DE)

(21) Appl. No.: 15/362,038

(22) Filed: Nov. 28, 2016

Related U.S. Application Data
(60) Provisional application No. 62/260,971, filed on Nov. 30, 2015.

(51) Int. Cl. A61N 7/00; B33Y 70/00; B33Y 10/00; B33Y 80/00; H02N 2/00; B06B 1/06

(52) U.S. Cl. CPC A61N 7/00; H02N 2/001; B06B 1/0644; B33Y 10/00; B33Y 80/00; B33Y 70/00; C01B 31/0446; A61N 2007/0017

(57) ABSTRACT

A system, method, and graphene device. The graphene devices includes one or more graphene layers configured to emit a wave to a site of a body of a user. The device further includes a frame housing the one or more graphene layers. The device further includes a driver connected to the one or more graphene layers to communicate an electronic signal to the one or more graphene layers that is converted to the wave. The device further includes a power source connected to the driver for powering the driver to generate the electronic signal.

### PRIORITY STATEMENT

This application claims priority to U.S. Provisional Patent Application No. 62/260,971, filed Nov. 30, 2015, and entitled “Graphene Based Ultrasound Generating Device System and Method”, hereby incorporated by reference in its entirety.

### BACKGROUND

I. Field of the Disclosure

The illustrative embodiments relate to portable electronic devices. More specifically, but not exclusively, the illustrative embodiments relate to a system, method, and device for utilizing graphene-based wearable devices for wound treatment.

II. Description of the Art

The hospitalization of chronically ill patients poses certain challenges for the caregiver. Many such bedridden patients suffer from significant co-morbidities that often complicate their ongoing care. Patients that cannot ambulate are at a significant risk for the development of pressure ulcers and other sores. Debilitating injuries and diseases may prolong the periods of bedrest increasing the probability of a patient suffering from pressure ulcers. In some cases, when skin or other tissues are injured, the body responds by facilitating the migration of fibroblasts. Fibroblasts are a type of connective tissue that facilitate would healing and patient recovery by synthesizing the extracellular matrix and collagen. Increasing wound healing to improve patient health and well-being while reducing complications is very important.

### SUMMARY OF THE DISCLOSURE

One embodiment provides a system, method, and graphene device. The graphene devices includes one or more graphene layers configured to emit a wave to a site of a body of a user. The device further includes a frame housing the one or more graphene layers. The device further includes a driver connected to the one or more graphene layers to communicate an electronic signal to the one or more graphene layers that is converted to the wave. The device further includes a power source connected to the driver for powering the driver to generate the electronic signal.

Another embodiment provides a graphene device. The device includes a frame supporting circuitry of the graphene device. The device further includes a graphene layer that converts first electronic signals to waves. The device further includes a driver that generates the first electronic signals. The device further includes a user interface controlling the electronic signals sent to the graphene layer including at least the frequency of the first electronic signals, an amplitude, and a time period the first electronic signals are generated. The device further includes a timer controlling the time period.

Yet another embodiment provides a method for forming a graphene device for treating wounds. One or more graphene layers are created. The one or more graphene layers are secured to a frame. The one or more graphene layers are connected to logic including at least a signal generator.

### BRIEF DESCRIPTION OF THE DRAWINGS

Illustrated embodiments of the present invention are described in detail below with reference to the attached drawing figures, which are incorporated by reference herein, and where:

FIG. 1 is a pictorial representation of graphene systems in accordance with an illustrative embodiment:

FIG. 2 is a pictorial representation of a graphene system in accordance with an illustrative embodiment;

FIG. 3 is a pictorial representation of a graphene system applied to a user in accordance with an illustrative embodiment;

FIG. 4 is a block diagram of a graphene system in accordance with an illustrative embodiment; and

FIG. 5 is a flowchart of a process for generating a graphene system in accordance with an illustrative embodiment.

### DETAILED DESCRIPTION OF THE DISCLOSURE

The illustrative embodiments provide a system, method, and graphene-based device for treating wounds with ultrasonic motion and emissions. In one embodiment, one or more layers of graphene are generated. The graphene layers may be layered as part of a mesh that is secured within a framework. In one embodiment, the framework is an adhesive packaging or dressing that may be secured to skin or tissue of a patient on or above a wound. The graphene layers may be driven to generate ultrasonic emissions by a battery, amplifier, drivers, or other circuitry. The graphene layers may also be driven by natural radio frequency noise in the environment to vibrate or emit radio frequency signals that enhance wound care and healing. As a result, pressure ulcers and other slow healing conditions may be addressed utilizing the graphene-based systems, devices, and methods herein described.

The illustrative embodiments provide a graphene device and system enhanced with a graphene layer. Graphene is an allotrope of carbon in the form of an atomic-scale, hexagonal lattice in which one atom forms each vertex. Graphene is about two hundred and seven times (207) stronger than steel by weight, conducts heat and electricity efficiently and is nearly transparent. The graphene layer provide lighter and smaller footprint devices that effectively generate ultrasonic signals and other waves for treating wounds. In one embodiment, the graphene layers may be positioned in sets or arrays that are tuned to distinct frequencies, wavelengths, or so forth to treat a specific wound.

In one embodiment, the graphene is formed in sheets that are then shaped into wound application sections. Graphene or graphene-like structure may also be formed. In one embodiment, the graphene layers are mounted, attached, or integrated into treatment systems. The graphene components are light, biocompatible, and easily inserted into the wound treatment system. The graphene may also be utilized to form the framework, structures, or various waveguide structures that more effectively communicate the ultrasonic waves. Waveguides may also be utilized to direct the signals. The waveguides are structures that guide waves, such as sound waves to propagate the signals with minimal loss of energy.

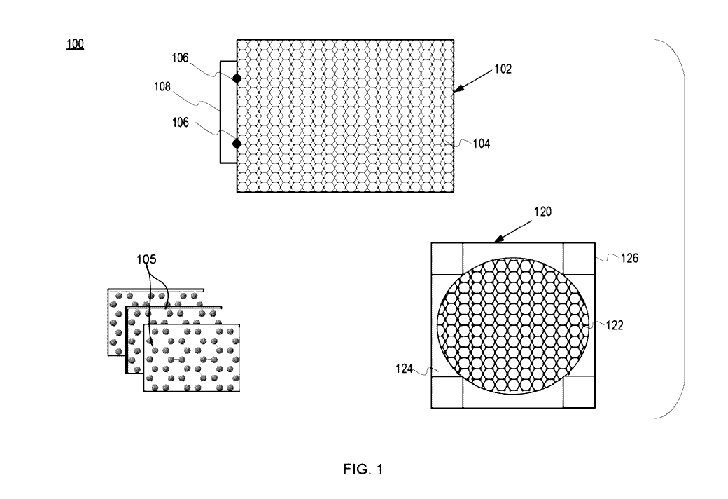

FIG. 1 is a pictorial representation of a graphene systems 100 in accordance with an illustrative embodiment. The graphene systems 100 herein described may have any number of components, configurations, formats, or structures. The graphene systems 100 may be utilized to treat wounds of different sizes and shapes. Wounds may represent any injury, sore, tissue, malady, or area that may benefit from treatment. The graphene device 102 includes a graphene layer 104, electrodes 106, and driver 108. The driver 108 may include one or more batteries, amplifiers, connectors, user interfaces, and other components for operating the graphene system 102.

The graphene device 120 may also include a graphene layer 122, a frame 124, and generator 126. The graphene layers 104 and 120 may be formed in part from one or more sheets 105 of graphene. In one embodiment, the graphene layers 104 and 120 may be used alone because of the lightweight, flexible, and inert properties. In other embodiments, the graphene layers 104 and 120 may be bound or integrated with a substrate or other materials. For example, the graphene layers 104 and 120 may be bound to atoms, compounds, or materials that are inert or have communication or healing properties.

In one embodiment, the sheets 105 of graphene may be layered, wrapped, stacked, folded or otherwise manipulated to form all or portions of the graphene devices 102 and 120. The sheets 105 may be created utilizing any number of processes (e.g., liquid phase exfoliation, chemical vapor/thin film deposition, electrochemical synthesis, hydrothermal self-assembly, chemical reduction, micromechanical exfoliation, epitaxial growth, carbon nanotube deposition, nano-scale 3D printing, spin coating, supersonic spray, carbon nanotube unzipping, etc.). Graphenite, carbon nanotubes, graphene oxide hydrogels, hyper honeycomb formed of carbon atoms, graphene analogs, or other similar materials may also be utilized to form portions of the graphene devices 102 and 120, such as graphene diaphragms represented by the graphene layers 104 and 122. The sheets 105 may also be utilized to form other portions of the graphene devices 102 and 120, such as hybrid batteries, the frame 124, or so forth.

The sheets 105 may be layered, shaped, and/or secured utilizing other components, such as metallic bands, frameworks, or other structural components. In one embodiment, layers of graphene (e.g., the sheets 105) may be imparted integrated, or embedded on a substrate or scaffolding that may remain or be removed to form a battery, frame, securing mechanisms, or one or more graphene structures of the graphene devices 102 and 120. In another embodiment, the sheets 105 may be reinforced utilizing carbon nanotubes or other structures. The carbon nanotubes may act as reinforcing bars (e.g., an aerogel, graphene oxide hydrogels, etc.) strengthening the thermal, electrical, and mechanical properties of the graphene layers 104 and 122 formed by the sheets 105.

In one embodiment, the sheets 105 of graphene may be soaked in solvent and then overlaid on an underlying substrate. The solvent may be evaporated over time leaving the sheets 105 of graphene that have taken the shape of the underlying structure. For example, the sheets 105 may be overlaid on a specially shaped frame 104 to form all or portions of the graphene layer 122, support structure, and/or electrical components of the graphene device 120. The sheets 105 may represent entire layers, meshes, lattices, or other configurations. For example, the sheets 105 may be pre-curved or otherwise shaped to fit specific body parts, areas, or so forth.

The graphene layers 102 and 122 may be driven to emit a particular radio frequency or to vibrate at a specified frequency. In one embodiment, the graphene layers 102 and 122 may be configured to emit ultrasonic signals that are therapeutic for a patient's wound or other treatment area. The graphene layers 104 and 122 are highly conductive and may be driven by a power source to waves of a specified amplitude and frequency or to vibrate at a specified resonance. In one embodiment, the graphene device 102 is powered by the driver 108. The driver 108 may be a housing that includes one or more batteries, signal generators, user interfaces (e.g., light emitting diodes, touch screens, dials, etc.) and amplifiers for driving the graphene layer 104. The driver 108 is connected to the graphene layer 104 by the electrodes 106. The graphene device 102 may include any number of electrodes 106, wires, or connection points to the driver 108 to drive the graphene layer 104.

In one embodiment, the driver 108 may include a touch screen or dial for adjusting the frequency of waves generated by the graphene layer 104. The size and shape of the graphene layer 104 may be configured to fit a particular wound or site. For example, the graphene layer 104 may be rolled or folded out to fit a specific size wound. In other embodiments, the graphene device 102 may be trimmed to a particular size and shape. The graphene device systems 100 may be configured to fit into one or more other wound dressings, bandages, reduced pressure treatment systems, or other treatment systems. As shown, the graphene devices 102 and 120 may be configured to fit different wound shapes and locations. The graphene devices 102 and 120 are lightweight to prevent further damage to the wound or applied site.

In one embodiment, the graphene layers 102 and 122 may correspond to disposable sections of the graphene systems 100. As a result, the graphene layers 102 and 122 may be disposed once utilized with new a graphene layer 104 attached to the driver 108 for the graphene device 102 and a graphene layer 122 attached to the frame 124 of the graphene device 120. As a result, the electronics of the graphene system 100 may be reused to conserve resources and save money.

The graphene systems 100 may also communicate with one or more networks, such as a personal area network, local area network, or so forth. In one embodiment, the graphene systems 100 may include transceivers or receivers for receiving instructions, commands, or other input from wireless devices or so forth. For example, a user may utilize a remote interface to adjust the frequency and amplitude of waves applied by the graphene systems 100. The graphene systems 100 may also include sensors and components for adjusting humidity level, perfusion level, temperature, gaseous emissions or pH levels, or so forth. In one embodiment, the graphene systems 100 may be configured to interconnect to expand the size and area that may be treated utilizing ultrasonic or other sound waves.

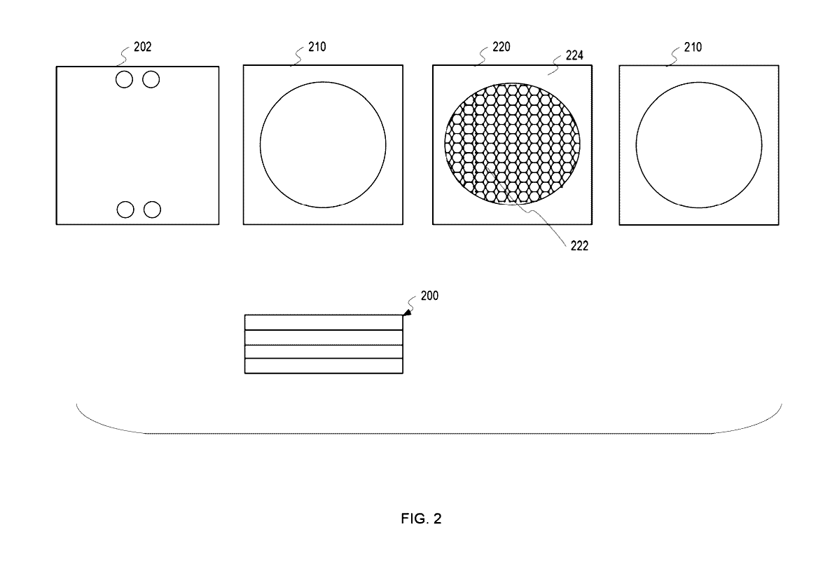

FIG. 2 is a pictorial representation of a graphene system 200 in accordance with an illustrative embodiment. In one embodiment, the graphene system 200 may include any number of layers or components. For example, the graphene system 200 may include a logic layer 202, spacer layers 210, and a graphene layer 220. The layers of the graphene system 200 may be deposited, stacked, or otherwise combined. In one embodiment, the layers may be between 1 mm-5 cm when stacked to form the graphene system 200. The depth of the layers may be greater or less based on the number and configuration of the layers.

In one embodiment, the logic layer 202 may include any number of electrodes, signal generators, batteries or power generators, amplifiers, processors, application-specific integrated circuits, chips, contacts, wires, traces, or other components for electrically connecting portions of the graphene system 200.

The spacer layers 210 may separate the logic layer 202 from the graphene layer 220. The spacer layers 210 may isolate the graphene layer 220 to further enhance the ultrasonic waves generated and prevent unwanted noise. In some embodiments, the spacer layers 210 may separate the graphene layer 220 from the wound providing space for the graphene layer 220 to operate to communicate ultrasonic waves. In some embodiments, the spacer layers 210 may not be utilized or may be a portion of the logic layer 202.

In one embodiment, an electrical signal is applied to the graphene layer 220. The graphene layer 220 includes a graphene transducer 222 secured by a frame 224. In one embodiment, the graphene transducer 222 is circularly shaped. However, in other embodiments the graphene transducer 222 may be elliptical, square, oblong, rectangular, hexagonal, or any number of other custom or pre-defined shapes. The graphene layer 220 may act as a driver for converting the electrical audio signals into radio frequency signals or sound waves that are directed at the wound or tissue site. The frame 224 may include a number of electrodes or contacts for applying an electrical signal to the graphene transducer 222 that is then converted to ultrasonic waves by the graphene transducer 222. For example, the electrodes may be positioned proximate a first and second side of the graphene membrane or diaphragm represented by the graphene transducer 222. In another example, the electrodes may be aligned along a single side of the graphene transducer 222.

In one embodiment, an electrical signal is applied to the graphene transducer 222 to generate sounds waves that are then propagated to a wound of the user. In some embodiments, the spacer layers 210 may act as wave guides for conducting or focusing the emissions of the graphene transducer 222. The graphene transducer 222 may be one or more layers of graphene that are layered to produce the desired ultrasonic waves. In other embodiments, the graphene transducer 222 may include other compounds, substrates or layers. Although not shown, the graphene system 200 may include a number of graphene diaphragms or transducers configured to generate sounds waves at distinct frequency ranges (e.g., bass, woofer, tweeter, midrange, etc.) or to vibrate at a specified frequency corresponding to the treatment being received by the user.

The frequency and amplitude may also be adjusted by a user or medical professional utilizing a user interface that may be integrated with the electrode layer 202. The performance of the graphene system 200 may be extremely energy efficient and effective based of the rigidity, conductivity, and weight properties of graphene. For example, in some tests graphene membranes, such as the graphene transducer 222, have been shown to efficiently convert 99% of the driving energy for the graphene system 200 to ultrasonic waves. The frequency responses are also sharp and more accurate than traditional forms of transducers, speakers, diaphragms, or emitting components. The high fidelity reproduction of waves of the graphene system 200 may be based on the frequency response characteristics of the graphene transducer 222.

The graphene system 200 may be configured for direct or indirect contact with the tissue of the user. For example, the vibrations or emissions of the graphene layer 220 may directly stimulate tissue regeneration and recover. Alternatively, the graphene layer 220 may be placed adjacent the tissue with the emissions providing beneficial effects to the tissue.

In one embodiment, the graphene system 200 may include the logic layer 202, a first spacer layer 210, a graphene layer 220, and followed by another spacer layer 210. The graphene system 200 may also include any number of housings for securing the layers or components of the graphene system 200 to each other.

All or portions of the graphene system 200 may be reusable. For example, the electrode layer 202 may be removed and then the spacer layers 210 and the graphene layer 220 that may come in contact with the user may be discarded or cleaned.

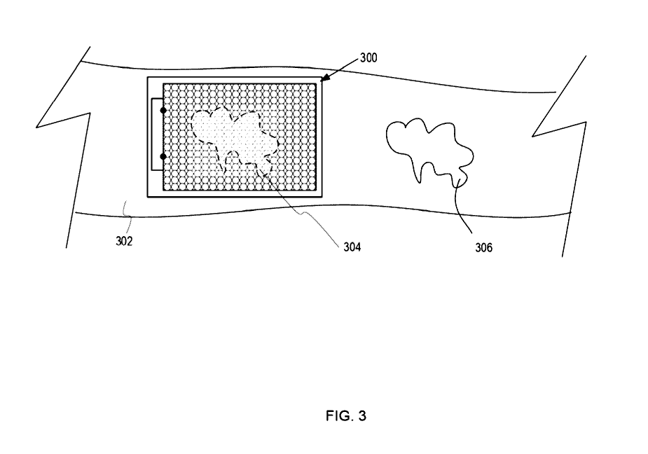

FIG. 3 is a pictorial representation of a graphene system 300 applied to a user in accordance with an illustrative embodiment. In one embodiment, the graphene system 300 is applied to an arm 302 of a user covering a wound 304. A second wound 306 is shown for illustrative purposes.

The graphene system 300 produces high frequency sound waves that travel deep into tissue, such as the wound 304. The graphene system 300 may be utilized to perform any number of ultrasonic procedures that utilize ultrasound for therapeutic benefit. This may include HIFU, lithotripsy, targeted ultrasound, drug delivery, transdermal ultrasound drug delivery, ultrasound, hemostasis, cancer therapy, and ultrasound assisted thrombolysis. For example, the ultrasonic waves generated by the graphene system 300 may be utilized to treat ligament sprains, muscle strains, tendinitis, joint inflammation, plantar fasciitis, metatarsalgia, facet irrigation, impingement syndrome, bursitis, rheumatoid arthritis, osteoarthritis, and scar tissue adhesion. In one embodiment, the graphene system 300 may be utilized to create gentle therapeutic heat. The ultrasonic waves may generate deep within body tissues for the treatment of selected medical conditions, such as pain, muscle spasms, wound healing, joint contractures, and so forth. In one embodiment, the graphene system 300 may include timers for alternating the amount of time the ultrasonic waves are applied. The graphene system 300 may be utilized alone or with a gel to reduce friction and assist in transmission of ultrasonic waves.

In other embodiments, the graphene system 300 may be utilized to generate images of a specific body part, segment, appendage or so forth. For example, the graphene system 300 may include a receiver that receives back reflected signals, waves, or so forth. As a result, the graphene system 300 may be able to generate any number of images (e.g., still images, video, etc.) for examining a particular area, site, or wound. In another embodiment, the graphene system 300 may have a secondary receiver that is positioned opposite the wound 304 to capture detailed tissue, bone, cellular, vein, blood flow, DNA, and other applicable site and patient information.

The graphene system 300 may be applied utilizing adhesives, straps, casts, clips, applicators, raps, bandages, tape, sutures, staples, or any number of temporary or permanent attachment mechanisms. The graphene system 300 may also be integrated with other treatment systems applied to the user.

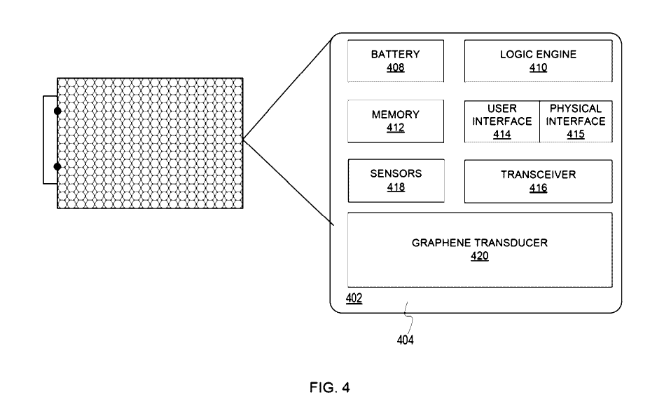

FIG. 4 is a block diagram of a graphene system 402 in accordance with an illustrative embodiment. In one embodiment, the graphene system 402 may enhance wound healing for a patient. For example, the graphene system 402 may provide ultrasonic, wave, RF emission, or vibration based therapy to stimulate wound recovery, tissue growth, and so forth utilizing the various properties of graphene.

As shown, the graphene system 402 may be a stand-alone device or may be physically or wirelessly linked to other graphene systems or one or more electronic devices, such as laptops, cellular devices, or so forth. Settings (e.g., frequency, amplitude, etc.), user input, and commands may be received through the graphene system 402 (or other externally connected devices).

In one embodiment, the graphene system 402 may include a frame 404, a battery 408, a logic engine 410, a memory 412, user interface 414, physical interface 415, a transceiver 416, sensors 418, and graphene transducer 420. The frame 404 is a lightweight and flexible structure for supporting the components of the graphene system 402. In one embodiment, the frame 404 is formed from graphene layers or other carbon structures. The frame 404 may also be composed of any number of other fabrics, polymers, plastics, composites, or other combinations of materials suitable for personal use by a user. In one embodiment, the frame 404 may include adhesives for securing the graphene system 402 in place during usage. The adhesives or other securing mechanisms of the frame may ensure that the graphene transducer 420 stays properly positioned.

The battery 408 is a power storage device configured to power the graphene system 402. In other embodiments, the battery 408 may represent a fuel cell, thermal electric generator, piezo electric generator, thermal generator, solar charger, ultra-capacitor, or other existing or developing power storage technologies. For example, the battery 408 may be an alternative power source that operates based on body heat, motion, extraneous RF signals, or other power sources.

The logic engine 410 is the logic that controls the operation and functionality of the graphene system 402. The logic may be utilized to refer to the electrical and power components of the graphene system 402 that are utilized to power the one or more graphene layers. In some embodiments, the logic engine 410 may be represented by a simple on/off switch or adjustment dial for increasing or decreasing the amplitude and frequency of the waves emitted by the physical interface 415 (e.g., graphene transducer 420 or diaphragm). In one embodiment, portions of the graphene system 402 may be interchangeable and utilized with fresh graphene transducers. The logic engine 410 acts as a driver to communicate signals to the graphene transducer 420. The logic engine 410 may include a signal generator, amplifier, and timer and other components for generating and communicating signals or patterns to the graphene transducer 420.

The logic engine 410 may also include circuitry, chips, or other digital logic. The logic engine 410 may also include programs, scripts, operating systems, applications, and instructions that may be implemented to operate the logic engine 410. The logic engine 410 may represent hardware, software, firmware, or any combination thereof. In one embodiment, the logic engine 410 may include one or more processors. The logic engine 410 may also represent an application specific integrated circuit (ASIC) or field programmable gate array (FPGA). The logic engine 410 may be utilize information from the sensors 418 to determine the biometric information, data, and readings of the user (e.g., temperature, pulse rate, blood oxygenation, etc.). The logic engine 402 may utilize this information and other criteria to adjust the settings of the graphene system 402. For example, the amplitude may be decreased or the therapy device represented by the graphene system 402 may be turned off in response to the user's pulse rate or temperature increasing dramatically (e.g., representation of pain, fever, fear, or so forth).

The logic engine 410 may also process user input to determine commands implemented by the graphene system 402. The user input may be determined by the sensors 418 or received from the user interface 414 to determine specific actions to be taken. In one embodiment, the logic engine 410 may implement changes to the amplitude, frequency, and time periods of the waves that are generated by the graphene layers.

In one embodiment, a processor included in the logic engine 410 is circuitry or logic enabled to control execution of a set of instructions. The processor may be one or more microprocessors, digital signal processors, application-specific integrated circuits (ASIC), central processing units, or other devices suitable for controlling an electronic device including one or more hardware and software elements, executing software, instructions, programs, and applications, converting and processing signals and information, and performing other related tasks. The processor may be a single chip or integrated with other computing or communications elements.

The memory 412 is a hardware element, device, or recording media configured to store data for subsequent retrieval or access at a later time. The memory 412 may be static or dynamic memory. The memory 412 may include a hard disk, random access memory, cache, removable media drive, mass storage, or configuration suitable as storage for data, instructions, and information. In one embodiment, the memory 412 and the logic engine 410 may be integrated. The memory 412 may use any type of volatile or non-volatile storage techniques and mediums. The memory 412 may store information related to the status or commands of a user, the graphene system 402, and/or other peripherals, such as a wireless devices, controllers, smart watches, and so forth. In one embodiment, the memory 412 may display instructions or programs for controlling the user interface 414 including one or more switches, dials, touch interfaces, LEDs or other light emitting components, speakers, tactile generators (e.g., vibrator), and so forth. The memory 412 may also store the user input information associated with each command. In one embodiment, a particular application or settings specified for a wound type may be stored in memory 412 and executed by the logic engine 410.

In one embodiment, the processor may execute instructions stored in the memory. For example, the processor may process commands for electrical signals into a format or electrical signals that may be generated by a driver (e.g., may be integrated with the logic engine 410), amplified, and converted by the graphene layer(s) of the graphene transducer 420 into waves. The graphene transducer 420 converts electrical signals received from the logic engine 410 into waves, signals, or vibration patterns that are applied to a patient wound or other site.

The transceiver 416 is a component comprising both a transmitter and receiver which may be combined and share common circuitry on a single housing. The transceiver 416 may communicate utilizing Bluetooth, Wi-Fi, ZigBee, Ant+, near field communications, wireless USB, infrared, mobile body area networks, ultra-wideband communications, cellular (e.g., 4G, 4G, 5G, PCS, GSM, etc.) or other suitable radio frequency standards, networks, protocols, or communications. The transceiver 416 may also be a hybrid transceiver that supports a number of different communications. For example, the transceiver 416 may communicate with a wireless device or other systems utilizing wired interfaces (e.g., wires, traces, etc.), NFC or Bluetooth communications. The transceiver 416 may allow the graphene system 402 to be turned on/off remotely. For example, an application of a smart phone may act as the user interface 414 allowing the user to control operation of the graphene system 402. The transceiver 416 may also include a component for receiving waves or signals generated by the graphene transducer 420 to perform imaging of a portion of a body of the user. For example, the graphene device 402 may be utilized to perform an ultrasound (e.g., vein issue, broken bone, baby in-utero, etc.) for the user. The graphene system 402 may also include a receiver only.

The components of the graphene system 402 may be electrically connected utilizing any number of wires, contact points, leads, busses, wireless interfaces, or so forth. In one embodiment, the frame 404 may include any of the electrical, structural, and other functional and aesthetic components of the graphene system 402. For example, the graphene device 402 may be fabricated with built in processors, chips, memories, batteries, interconnects, and other components that are integrated with the frame 404 and removable attached to the graphene transducer 420 (e.g., may be replaced after use on a patient). For example, semiconductor manufacturing processes may be utilized to create the graphene device 402 as an integrated and more secure unit. As a result, functionality, security, shock resistance, waterproof properties, and so forth may be enhanced.

In addition, the graphene system 402 may include any number of computing and communications components, devices or elements which may include busses, motherboards, circuits, chips, sensors, ports, interfaces, cards, converters, adapters, connections, transceivers, displays, antennas, and other similar components. The additional computing and communications components may also be integrated with, attached to, or part of the frame 404. The physical interface 415 is the hardware interface of the graphene system 402 for connecting and communicating with wireless devices, medical control systems, or other electrical components.

The physical interface 415 may include any number of pins, arms, or connectors for electrically interfacing with the contacts or other interface components of external devices or other charging or synchronization devices. For example, the physical interface 415 may be a micro USB port for charging the battery 408 and providing instructions to the logic engine 410. In another embodiment, the physical interface 415 may include a wireless inductor for charging the graphene system 402 without a physical connection to a charging device.

The user interface 414 is a hardware and/or software interface for receiving commands, instructions, or input through the touch (haptics) of the user, voice commands, or predefined motions. The user interface 414 may be utilized to control the other functions of the graphene system 402. The user interface 414 may include an LED array, one or more touch sensitive buttons or portions, a miniature screen or display, a dial, switch, or other input/output components. The user interface 414 may be controlled by the user or based on commands received from an external device or a linked wireless device. The user interface 414 may also receive commands, feedback, or a user profile for operating without direct instructions from the user.

In one embodiment, the user may provide feedback by tapping the user interface 414 once, twice, three times, or any number of times. Similarly, a swiping motion may be utilized across or in front of the user interface 414 (e.g., the exterior surface of the graphene system 402) to implement a predefined action. Swiping motions in any number of directions may be associated with specific activities, such as increase the amplitude, change the frequency, and change the time period for activating and deactivating the graphene system 402 to generate waves. The user interface 414 may include a camera or other sensors for sensing motions, gestures, or symbols provided as feedback or instructions.

The sensors 418 may include pulse oximeters, accelerometers, gyroscopes, magnetometers, inertial sensors, photo detectors (e.g., spectroscopy), miniature cameras, humidity sensors, temperature sensors, chemical sensors (e.g., determine pH levels, compounds, and concentrations) and other similar instruments for detecting location, orientation, motion, wound condition, and so forth. The sensors 418 may also be utilized to gather optical images, data, and measurements and determine the wave intensity applied.

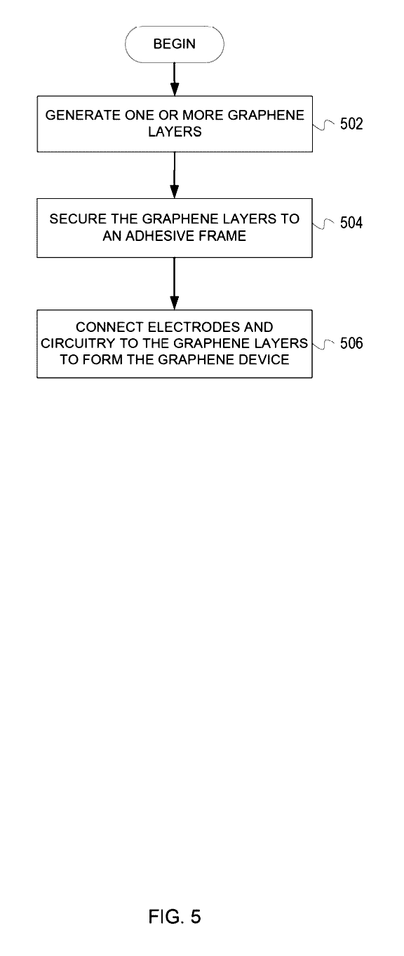

FIG. 5 is a flowchart of a process for generating a graphene system in accordance with an illustrative embodiment. The process of FIG. 5 may be implemented utilizing any number of devices, systems, equipment, facilities, or so forth (referred to generically as a “system”). For example, semiconductor manufacturing facilities and processes (or analogs) may be utilized. The process may be implemented automatically, semi-automatically, manually, or any combination thereof. For example, the process of FIG. 5 may be implemented to generate a graphene system or device.

The process may begin by generating one or more graphene layers (step 502). The graphene layers may be generated one at a time (or utilizing another carbon structure or material). The graphene layers may be generated utilizing any number of processes or in any number of environments, such as chemical vapor deposition, epitaxial growth, nano-3D printing, or the numerous other methods being developed or currently utilized. In one embodiment, the graphene layers may be generated on a substrate or other framework that may make up one or more portions of a wound dressing. The graphene layers may also include any number of anti-bacterial coatings, antibiotics, antifungals, or other medicinal coatings that may be applied to the wound

Next, the system secures the graphene layers to an adhesive frame (step 504). In one embodiment, a single graphene layer may be positioned on a frame. For example, the graphene layer may be positioned over a frame or structure of the graphene device. The frame may include adhesives for gently securing the graphene device over a wound site. In another embodiment, the graphene device may include straps, clips, or other components for positioning the graphene device. The graphene layer may be mechanically, chemically, or otherwise bound to a frame that makes up the one or more graphene layers. During step 504, the graphene layer may also be trimmed or otherwise shaped to a desired shape and size. For example, different types of wounds may require different sizes and shapes of graphene layers. In another embodiment, the graphene layers may be layered on top of each other or otherwise positioned. In one embodiment, graphene layers may be bonded to another substrate or material to enhance the effectiveness of the graphene at emitting a specified radio frequency, such as ultrasonic sound waves. The graphene layers may also be configured to vibrate to stimulate cell growth and recover.

Next, the system connects electrodes and circuitry to the graphene layers to form the graphene device (step 506). The adhesive frame may include circuitry for operating the graphene device including a power source (e.g., battery, solar cell, fuel cell, piezo electric generator, etc.), signal generator, amplifier, or so forth. The circuitry may be connected to the circuitry utilize electrodes. The graphene device may also include one or more electrode layers, spacers, supporting frames, and so forth. In one example, the graphene layers may be generated and layered utilizing semiconductor manufacturing processes. In one embodiment, wires or leads (e.g., gold wires, integrated traces, etc.) may be connected to electrodes of the graphene layers to convert electronic signals to radio frequency signals, sound waves, or vibration patterns for the graphene layers. The one or more layers of the graphene device may be mechanically, structurally, or chemically secured together. The graphene may be produced in sheets, meshes, or framework.

In another embodiment, the graphene device may include a method for utilization. The graphene device may be secured over a wound or other site of a body of a user. For example, the graphene device may include an adhesive frame that is used to securely position the graphene device at the desired location. In other embodiments, the graphene device may be positioned with an article of clothing (e.g., gown, jacket, single arm or leg sleeve, pants, hat, etc.), fabric, clips, or other securing mechanisms.

The graphene device may be activated. The graphene device may be activated automatically or based on a user selection. For example, the user interface may include an on/off selection element (e.g., switch, touch interface, dial, etc.). In one embodiment, the graphene device draws power from the user or environment to generate waves that are applied to the site. In other embodiments, the graphene device may be powered by a power source, such as a battery, fuel cell, solar cells, piezo electric generator, dynamo, thermal converter or so forth.

The graphene device generates waves applied to the wound based on an electrical signal that is generated by the internal components and circuitry. In one embodiment, the graphene device may also receive and process reflected or original waves to perform imaging and analysis, such as ultrasounds, X-rays, or so forth.

The illustrative embodiments are not to be limited to the particular embodiments described herein. In particular, the illustrative embodiments contemplate numerous variations in the type of ways in which embodiments may be applied. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A graphene device, comprising:
one or more graphene layers configured to emit a wave to a site of a body of a user;
a frame housing the one or more graphene layers;
a driver connected to the one or more graphene layers to communicate an electronic signal to the one or more graphene layers that is converted to the wave; and
a power source connected to the driver for powering the driver to generate the electronic signal.

2. The graphene device of claim 1, wherein the one or more graphene layers form a graphene diaphragm for converting the electronic signal to the wave.

3. The graphene device of claim 2, wherein the graphene diaphragm is printed utilizing a three dimensional printer.

4. The graphene device of claim 1, wherein the wave is an ultrasonic wave, and wherein the site is a wound.

5. The graphene device of claim 1, further comprising:
a user interface controlling the driver to adjust the frequency and amplitude of the electronic signal.

6. The graphene device of claim 5, wherein the user interface is a touchscreen for adjusting the frequency, amplitude, and time periods applied to the one or more graphene layers.

7. The graphene device of claim 1, wherein the driver and the power source are integrated with the frame.

8. The graphene device of claim 1, wherein the wave causes the one or more graphene layers to vibrate to treat the site.

9. The graphene device of claim 1, wherein the power source is one or more of a battery, solar cells, a piezo electric generator, and a fuel cell.

10. The graphene device of claim 1, wherein the frame includes an adhesive for securing the frame to the body of the user.

11. A graphene device comprising:
a frame supporting circuitry of the graphene device,
a graphene layer that converts first electronic signals to waves;
a driver that generates the first electronic signals;
a user interface controlling the electronic signals sent to the graphene layer including at least the frequency of the first electronic signals, an amplitude, and a time period the first electronic signals are generated; and
a timer controlling the time period.

12. The graphene device of claim 11, wherein the frame includes an adhesive for securing the frame to a body of a user.

13. The graphene device of claim 11, wherein the waves are ultrasonic waves

14. The graphene device of claim 13, wherein the ultrasonic waves are utilized to image a portion of a body of a user.

15. A method for forming a graphene device for treating wounds, comprising:
creating one or more graphene layers;
securing the one or more graphene layers to a frame;
connecting the one or more graphene layers to logic including at least a signal generator.

16. The method of claim 15, further comprising:
connecting at least an electrode layer and a spacer layer to the graphene layers.

17. The method of claim 15, wherein the graphene layers are printed utilizing a three dimensional printer.

18. The method of claim 15, wherein the graphene layers convert electronic signals generated by the driver to ultrasonic waves for treating a wound.

19. The method of claim 15, wherein the logic further includes an amplifier for amplifying an electronic signal generated by the driver.

20. The method of claim 15, wherein the logic further includes a user interface for adjusting waves generated by the one or more graphene layers.
